(19) United States
(12) Patent Application Publication
(10) Pub. No.: US 2014/0220101 A1
(43) Pub. Date: Aug. 7, 2014

(54) SUBSTANTIALLY DRY NICOTINAMIDE PADS FOR TREATMENT OF SKIN INFLAMMATION

(71) Applicant: Bharat Janardan Sanyal, North Brunswick, NJ (US)

(72) Inventor: Bharat Janardan Sanyal, North Brunswick, NJ (US)

(21) Appl. No.: 14/171,514

(22) Filed: Feb. 3, 2014

Related U.S. Application Data
(60) Provisional application No. 61/761,353, filed on Feb. 6, 2013.

(51) Int. Cl. A61K 9/70; A61K 31/455

(52) U.S. Cl. CPC A61K 9/7007; A61K 31/455
USPC 424/443; 514/355

(57) ABSTRACT

A substantially dry nonwoven pad is provided containing a formulation comprising nicotinamide in an amount between about 1% to about 10% w/w based on the total weight of the formulation and at least one water-soluble gritty polymer in an amount between about 10% to about 75% w/w based on the total weight of the formulation. The nicotinamide-containing pad is used topically for treatment of skin inflammation, in one easy step. In a method for treating skin inflammation, including acne, rosacea, and acne scars (hyperpigmentation), the nicotinamide-containing pad is moistened, then applied to the skin treatment area using gentle scrubbing, followed by rapid absorption of the formulation by the skin, and rinsing the skin with water.

This application claims the benefit of earlier filed U.S. Provisional Application No. 61/761,353, filed on Feb. 6, 2013, which is hereby incorporated by reference herein.

### FIELD OF THE INVENTION

The present invention relates to use of substantially dry nicotinamide-containing pads in providing a one-step application for relief and/or treatment of skin inflammations resulting from acne and rosacea, removal of oily residues (caused by excess sebum production), opening of clogged pores, reducing/diminishing presence of skin hyperpigmentation (acne scars), skin exfoliation, among other uses. The substantially dry pads may contain between about 1%-10% w/w nicotinamide.

### BACKGROUND

Most known acne treatments rely on multi-step treatment regimes. For example, one step to cleanse, a second step to apply anti-acne medication (salicylic acid or benzoyl peroxide derivative, an antibiotic, or variant thereof), and a third step for toner/moisturizer. Some treatments include one step application of ointment, creams or lotions. However, ointment, aqueous based creams and lotions are not particularly effective in penetrating oily residues or secretions under which acne bacteria may reside or multiply.

Patient compliance is also an issue since some regimens require users to wait in between steps for optimal results. Because of time constraints, users often neglect following the steps or procedure as directed and experience a lack of results. Product contamination is also a significant issue that plagues the known techniques. This results when users dab the product on an applicator such as cotton swab, apply it on the acne area and then use the same potentially contaminated swab to get more product from the container, thus spreading contagion.

Most acne treatments fall under the following categories: over-the-counter (OTC) products, prescription products, and dermatological interventions. OTC products include mostly ointment, creams and lotions/liquids containing salicylic acid, benzoyl peroxide etc. Some contain herbal extracts or oils. These products offer limited value in terms of getting acne under control and can be time-consuming to apply and use, especially if multiple steps are involved. Travel with lotions and liquids can be difficult. Also, liquids and creams deteriorate quickly when the product is stored under non-optimal storage conditions. Proper storage conditions can then become a drawback. Peroxide solutions typically bleach clothes during a spill or misapplication—a common user complaint.

Prescription treatments typically include antibiotic products like clindamycin, tetracyclines, erythromycin, doxycycline, and the like. Retinoid derivatives such as isotretinoin (Accutane or Roaccutane) are frequently prescribed as well. Duration of use is limited due to side-effects. To minimize side-effects, patients need to take a break from antibiotics and do invasive tests—blood tests, liver function tests, etc. In some cases acne bacteria becomes resistant and doctors increase dosages and/or change medication. Acne then may recur with even more vigor and becomes harder to overcome.

For example, Nicomide™ (niacinamide tablets) require a patient to ingest large amounts of niacinamide which is subjected to the “first-pass effect”. This has the potential for side-effects in some individuals. In any case a tiny amount of the active ingredient actually makes it to the site where it is needed. Most of the active ingredient is excreted out.

Visits to a dermatologist can result in the most expensive treatment options, including laser treatment and dermabrasion. Patients often are anesthesized (local or general) depending upon the area to be treated and the severity of the problem. Often the treated skin is sore and is prone to further infection.

All of the above treatments are expensive and often bring along side-effects and health complications.

If a cleansing product could be found that was more simple to use in order to prevent and/or treat acne and rosacea related skin inflammation and skin hyperpigmentation, this would represent a useful contribution to the art.

If a cost-effective and a highly potent and efficacious way were available to prevent and/or treat acne or rosacea related skin inflammation and skin hyperpigmentation, this would also represent a useful contribution to the art.

Those experienced in pharmaceutical formulation are aware that patient non-compliance is a leading cause of ineffective treatments. After a while, patients get tired of following these time consuming multi-step treatments, take shortcuts or make omissions, and consequently see no results.

If a cleansing product could be found containing nicotinamide that helps users to address one or more of: eliminate oily residues on skin (excess sebum production caused by hormonal changes), open clogged skin pores, expose acne bacteria to nicotinamide for fast effect, moisturize skin, minimize acne scars (skin hyperpigmentation), this would represent a useful contribution to the art.

If a method for treating acne or rosacea related skin inflammation and skin hyperpigmentation were available to rapidly eliminate oily residues on skin (excess sebum production caused by hormonal changes), open clogged skin pores, expose acne bacteria to nicotinamide for fast effect, moisturize skin, minimize acne scars (skin hyperpigmentation), this would represent a useful contribution to the art.

### SUMMARY OF THE INVENTION

In one embodiment, a substantially dry pad includes nicotinamide. The substantially dry pads are made using nonwoven or woven materials which are also disposable and may be biodegradable. The substantially dry pads may contain a formulation including between about 1%-10% w/w nicotinamide based on the total weight of the formulation.

In an embodiment, a woven or nonwoven pad contains a formulation comprising nicotinamide in an amount between about 1% to about 10% w/w based on the total weight of the formulation, at least one water-soluble gritty polymer in an amount between about 10% to about 75% w/w based on the total weight of the formulation, wherein the pad is substantially dry.

In another embodiment, a method of treating skin inflammation, including acne and rosacea, comprises the steps of:

(a) providing a woven or nonwoven pad containing a formulation comprising nicotinamide in an amount between about 1% to about 10% w/w based on the total weight of the formulation, at least one water-soluble gritty polymer in an amount between about 10% to about 75% w/w based on the total weight of the formulation, wherein the pad is substantially dry;

(b) moistening the pad with water;

(c) applying the moistened pad to an area of skin in need of treatment;

(d) gently scrubbing the area of skin in need of treatment to apply the formulation to the skin;

(e) allowing the formulation to be absorbed into the skin for a period of at least about 20 sec.;

(f) removing the pad from the skin; and

(e) rinsing the skin with water.

In another embodiment, the method of treating skin inflammation may further include the steps of: providing a second pad as in the above method; moistening the second pad with water; pressing the second moistened pad to the area of skin in need of treatment in such a manner that the pad adheres to the skin; allowing the adhered pad to dry on the skin; removing the adhered pad from the skin; and rinsing the skin with water.

### BRIEF DESCRIPTION OF THE DRAWINGS

FIG. 1 depicts a high resolution photograph of the front or top surface of one embodiment (F101) of a substantially dry nonwoven pad containing and/or coated with a formulation comprising nicotinamide in an amount between about 1% to about 10% w/w based on the total weight of the formulation, at least one water-soluble gritty polymer in an amount between about 10% to about 75% w/w based on the total weight of the formulation. The at least one water-soluble gritty polymer has an average particle size of from about 50 microns to about 200 microns.

FIG. 2 depicts a photograph of the back or bottom surface of the substantially dry nonwoven pad of FIG. 1.

FIG. 3 depicts a photograph of a top plan view of the substantially dry nonwoven pad of FIG. 1. Each gradation shown is 1 mm.

FIG. 4 depicts a photograph of a top plan view of the substantially dry nonwoven pad in accordance with FIG. 1 wherein the scale is ruled in cm and inches.

FIG. 5 depicts a high resolution photograph of the front or top surface of another embodiment (D103) of a substantially dry nonwoven pad containing and/or coated with a formulation comprising nicotinamide in an amount between about 1% to about 10% w/w based on the total weight of the formulation, at least one water-soluble gritty polymer in an amount between about 10% to about 75% w/w based on the total weight of the formulation. This formulation produces a smoother surface with generally no visible granules.

FIG. 6 depicts a photograph of the back or bottom surface of the substantially dry nonwoven pad of FIG. 5.

FIG. 7 depicts a high resolution photograph of the front or top surface of another embodiment (A100) of a substantially dry nonwoven pad containing and/or coated with a formulation comprising nicotinamide in an amount between about 1% to about 10% w/w based on the total weight of the formulation, at least one water-soluble gritty polymer in an amount between about 10% to about 75% w/w based on the total weight of the formulation.

FIG. 8 depicts a photograph of the back or bottom surface of the substantially dry nonwoven pad of FIG. 7.

FIG. 9 depicts a high resolution photograph of the front or top surface of one embodiment of a substantially dry uncoated nonwoven pad (control).

FIG. 10 depicts a photograph of the back or bottom surface of the substantially dry nonwoven pad of FIG. 9.

### DETAILED DESCRIPTION

In the following description, the weight/weight (“w/w”) percentages generally represent dry solids or components in the formulation not including the weight of a woven or nonwoven substrate, matrix, or pad. It is understood that the formulations as described herein are applied to, coated onto, adhered to, or generally and/or substantially impregnated within, a woven or nonwoven substrate, matrix, or pad. The pad may be a medicated pad.

The term “substantially dry” as used herein means that the product is substantially free of water and generally feels dry to the touch. The products of the present invention comprise less than about 10% by weight of water, preferably than about 5% by weight of water, and more preferably less than about 1% by weight of water, the foregoing measured in a dry environment, e.g., low humidity. One of ordinary skill in the art would recognize that the water content of a product such as in the present invention can vary with the relative humidity of the environment.

The term “gritty” as used herein means a physicochemical property that provides frictional scrubbing or abrasion. For example, a gritty polymer may provide gentle cleansing of skin without producing excessive abrasion. The abrasive properties of a given gritty solid polymer are generally related to particle size and/or particle size distribution. A generally water-soluble polymer granule may have a water-soluble grittiness which helps scrub away oily residues and dead skin cells on acne prone skin without excessive abrasion. In general, micronized particle sizes of PVP and PEG2M produce less grittiness than other materials, while providing the appropriate degree of grittiness. In one embodiment, an average polymer particle size of about 150 microns may be used to produce a desirable grittiness in the final product. Particle sizes used to make the final product can range from about 50 microns to about 200 microns. The term “grittiness” refers to a physical quantity of gritty particles (by weight or volume), or degree of abrasiveness, or other related properties of a gritty material, which can be measured or determined by a user or observer.

In an embodiment, a substantially dry pad may contain between about 1%-25% w/w nicotinamide in a formulation optionally in combination with oils, emollients, and additives.

In another suitable embodiment, a substantially dry pad may contain between about 1%-10% w/w nicotinamide optionally in combination with oils, emollients, and additives.

For the purposes of the present disclosure, the term “nicotinamide” means nicotinic acid amide or niacinamide (CAS No. 98-92-0). Also contemplated are nicotinamide derivatives, including modified niacinamide, derivatized niacinamide, etc., including derivatives chemically bonded to a substrate. For example, one useful derivative is nicotinamide adenosine dinucleotide (NAD, CAS No. 53-84-9). Another useful compound is niacin (CAS No. 59-67-5), which comprises nicotinic acid, and is also known as Vitamin B3. It is understood that the present disclosure contemplates combinations of these derivatives, analogues thereof, and other related compounds, in addition to formulations containing a single representative species.

In another embodiment, a woven or nonwoven pad is provided containing a formulation comprising nicotinamide in an amount between about 1% to about 10% w/w based on the total weight of the formulation, and at least one water-soluble gritty polymer in an amount between about 10% to about 75% w/w based on the total weight of the formulation, wherein the pad is substantially dry.

In a further embodiment, a woven or nonwoven pad is provided containing a formulation comprising nicotinamide in an amount between about 1% to about 10% w/w based on the total weight of the formulation, at least one water-soluble gritty polymer in an amount between about 10% to about 75% w/w based on the total weight of the formulation, and an emollient oil, wherein the pad is substantially dry.

A method of making the medicated pad is described herein.

First, a suitable nicotinamide-containing formulation is prepared. Second, a woven or nonwoven substrate, matrix, membrane, film, or pad is coated with the formulation. Finally, the coated pad is dried to remove water using standard techniques.

Further, a method of using the medicated pad is described for the treatment and/or prevention of acne, rosacea, and other inflammatory skin condition. In its principal embodiment, the medicated pad is used topically as applied to the surface of human skin.

Acne is a rite of passage for teenagers. Pregnancy acne and adult acne is becoming more commonplace due to the stressful environment and times. Current treatment options include use of antibiotics (taken internally and applied topically), OTC ointment, lotions and creams (e.g., benzoyl peroxide, salicylic acid), and herbal remedies (e.g., aloe vera, tea tree oil, green tea).

Studies have shown that topical application of nicotinamide solution is more effective than antibiotics like Clindamycin (applied topically) when dealing with acne. Shalita A.R., et al., “Topical Nicotinamide compared with Clindamycin gel in treatment of inflammatory acne vulgaris,” Int. J. Dermatol. (1995) 34(6):434-437. There have been other marketed nicotinamide and/or niacinamide products—e.g. Nicomide™ (niacinamide tablets) for dealing with acne. Most commercial products are multi-step which include—face wash, acne treatment, skin toner and moisturizer. (Niacinamide is also known as nicotinamide or Vitamin B3.)

Niacinamide (Vitamin B3) USP grade is utilized for formulation. This material is available from a wide range of commercial suppliers, e.g., Parchem, Rochelle, N.Y.; Prinova, Carol Stream, Ill.

Accordingly, a one-step application solution embodied in the substantially dry nicotinamide-containing pad has been discovered for pore-cleansing, removal of oily residues (excess sebum production caused by hormonal changes), dealing with acne bacteria, and additionally skin moisturizing. The substantially dry nicotinamide-containing pad is a dry woven or nonwoven pad (e.g., Niapads™, available from Saiom Technologies Inc., North Brunswick, N.J.).

The substantially dry nicotinamide-containing pad prepared in accordance with the present invention can be used effectively for the following purposes and results.

a. Scrub away excess sebum, oily residues and open clogged skin pores.

b. To bring nicotinamide into contact with, or cause to interact with, acne bacteria and provide acne relief and/or treatment.

c. Moisturize skin with skin-compatible, skin-conditioning, and/or skin friendly emollients such as lavender oil.

d. Promote skin repair and healing, thus minimizing acne scars (skin hyperpigmentation).

e. Provide relief and/or treatment for acne and rosacea related skin inflammation.

f. Reduce skin hyperpigmentation resulting from acne and rosacea related inflammation.

g. Exfoliation, or removal of dead skin cells, resulting from scrubbing action.

h. Increased skin hydration—for minimizing skin wrinkles

i. Remove sun spots (melisma)—minimizing skin hyperpigmentation, even out skin tone.

j. Spot treatment capability. A user may cut a piece of the pad, slightly larger than the pimple, moisten it with water and place it on the pimple. When the pad dries up, simply peel it and rinse the skin surface.

Furthermore, the substantially dry pad containing nicotinamide is cost-effective, travel friendly, and has a long shelf life.

The substantially dry pad containing nicotinamide also assists in diminishing or reducing skin hyperpigmentation. In addition to providing relief from acne it also reduces acne scars. For example, Niacinamide assists in reducing skin hyperpigmentation based on known scientific literature. This feature of being able to take care of acne and acne scars in one step, is considered to be one of a kind.

The substantially dry pad containing nicotinamide embodies a novel product that may be used for skin cleansing, cosmetic, cosmeceutical, nutraceutical, and pharmaceutical applications. The substantially dry pad is a coated woven or nonwoven fabric pad made of fibers selected from cotton, wood pulp, hemp, jute, flax, acrylics, nylons, polyesters, polypropylenes, polyethylenes, polyvinyl acetates, polyurethanes, rayon, silks, keratins, celluloses, acetates, acrylics, cellulose esters, modacrylics, polyamides, polyesters, polyolefins, polyvinyl alcohols, or mixtures thereof.

Nonwoven materials are preferred substrates for the nicotinamide-containing pads. Suitable nonwoven fabrics include spun bond (‘S’), melt blown (‘M’), or combinations thereof (e.g., SM, SMS materials). Other useful nonwovens include needlepunch, spunlace, and the like. Appropriate films, membranes, and/or matrices are contemplated as useful substrates.

Furthermore, nonwoven materials may include treated or untreated materials, occlusive or non-occlusive materials, materials derived from synthetic or natural origin, blends, wools, acrylic, cotton, rayons, nylons, polyesters, and other fiber blends.

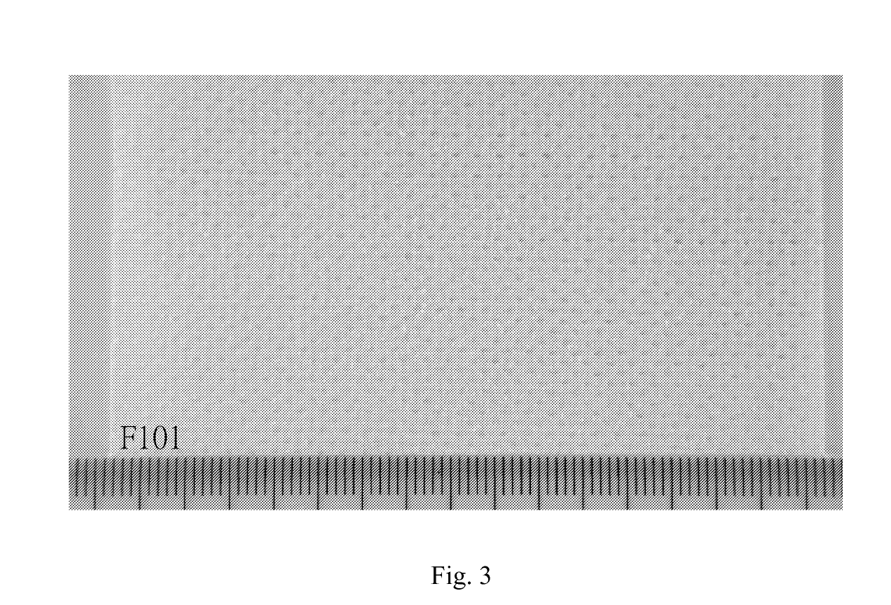

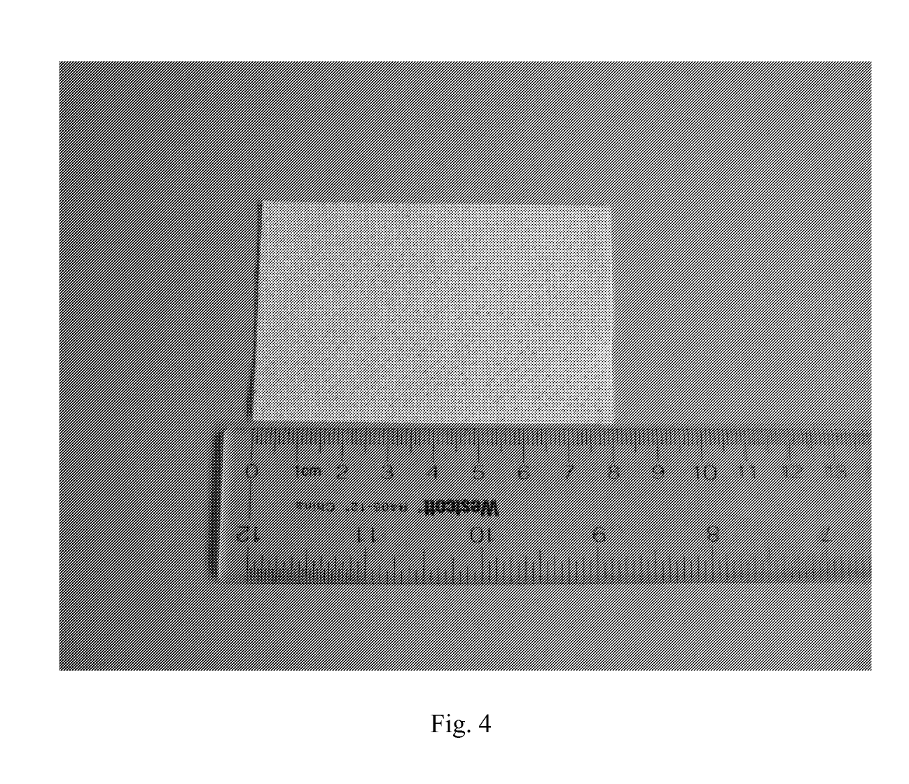

One useful nonwoven material for the substantially dry pads is spunlace aperture nonwoven 100% polyester fiber (also available in blends with absorbent cellulose or rayon with polyester fiber), which is available from a wide variety of commercial suppliers. The pad thickness can be from about 0.01 mm to about 100 mm. A preferred pad thickness can be between about 0.1 mm to about 5 mm. Exemplary substantially dry pads containing nicotinamide and at least one water-soluble gritty polymer are shown in FIGS. 3 and 4.

The substantially dry pad may contain nicotinamide in a range of about 1%-25% w/w (as a percentage of the total formulation). In another suitable embodiment, the substantially dry pad may contain nicotinamide in a range of about 1%-25% w/w. In another suitable embodiment, the substantially dry pad may contain nicotinamide in a range of about 1%-10% w/w. In a preferred embodiment, the substantially dry pad may contain nicotinamide in a range of about 4%-5% w/w.

The substantially dry pad including nicotinamide has a moisture content of <5%. In a preferred embodiment, the substantially dry pad including nicotinamide has a moisture content of <1%, and a water activity (a_{w})<0.5.

The substantially dry pad may contain a water-soluble and/or water-dispersible polymer component. Suitable water-soluble and/or water-dispersible polymers may include, but are not limited to, polyvinyl pyrrolidine (PVP), polyethylene glycol (PEG), ethyl cellulose, and derivatives or combinations thereof. The suitable water-soluble and/or water-dispersible polymers are provided as solid micronized particles or beads which are gently abrasive to human skin surfaces. These polymers are included in the formulations to provide grit or grittiness to the pad, thus providing gentle cleansing of skin without producing excessive abrasion. See, Kadajji, et al., “Water Soluble Polymers for Pharmaceutical Applications,” Polymers (2011) 3: 1972-2009; and Boregowda, et al., “Application of water-soluble/dispersible polymeric carriers in drug dissolution modulation,” Asian J. Pharm. Sci. (2011) 6(1): 26-35; both incorporated by reference herein.

Water-soluble polymers cover a wide range of highly varied families of products of natural origin (e.g., derived from plants, such as cellulose, or from sea animals, such as chitin) or synthetic origin. Natural polymer materials may be obtained from natural sources with or without processing and with or without additional modification. Synthetic polymers may be obtained from synthetic sources or by modification of products obtained from natural sources. These can be charged or neutral (ionic or non-ionic), branched, cross-linked, unbranched, modified, or derivatized natural polymers or synthetic polymers. These can be partially water-soluble, or soluble in presence of a co-solvent. The polymers can include blends, extrudates, block co-polymers, modifications such as cross-linking, and other suitable processing.

Suitable natural polymers can include, but are not limited to: polysaccharides (e.g., xanthan, carrageenan, guar, starches (including starch based derivatives, amylose), dextrans, pectins, hyaluronic acid); modified cellulose (e.g., ethers, acetates), hydroxypropyl methyl cellulose (HPMC), hydroxypropyl cellulose (HPC), hydroxyethyl cellulose (HEC), sodium carboxy methyl cellulose (Na-CMC); chitin, chitosan, or modified chitosan; gelatin; pectins (high-methoxy pectins, low-methoxy pectins); dextrans crosslinked with methacrylate or hydroxyl ethyl methacrylate; carrageenan-carboxymethyl cellulose blends; hyaluronic acids (HA), hyaluronic acid-alginic acid derived blends; alginates; polaxamers, which are nonionic triblock copolymers composed of a central hydrophobic chain of polyoxypropylene (poly(propylene oxide)) flanked by two hydrophilic chains of polyoxyethylene (poly(ethylene oxide)), such as hyaluronan-methylcellulose (HAMC); and the like. See, Rinaudo, “Chitin and chitosan: Properties and applications,” Prog. Polym. Sci. (2006) 31: 603-632; and Shaji, et al., “Chitosan: a novel pharmaceutical excipient,” Int. J. Pharmaceut. Appl. Sci. (2010) 1(1): 11-28; both incorporated by reference herein.

Synthetic polymers may be prepared or processed in many ways, including addition polymerization, condensation polymerization, and can include blends, extrudates, block co-polymers, modifications such as cross-linking, and other suitable processing. Structurally, synthetic polymers can be linear, branched chain, cross-linked, or networked. Synthetic polymers can possess one or more advantageous properties suitable for the nonwoven pads made according to the embodiments described herein, including elastomeric properties, fiber characteristics, thermoplastic properties, and biodegradability, to name a few. Synthetic polymers can include, but are not limited to: polyvinyl pyrrolidine (including PVP vinyl acetate, PVP-VA); polyacrylamides; polyacrylates or polyacrylic acid (PAA); polyacrylic acid copolymers, e.g., PAA with blocks of polyethylene oxide (PEO) and/or Polypropylene oxide (PPO); hydrophobically modified polyacrylic acid (HMPAA); polyethylene glycol (PEG); N-(2-hydroxypropyl)methacrylamide (HPMA); divinyl ether maleic anhydride (DIVEMA); polyoxazolines; polyphospho esters (e.g., polyphosphates, polyphosphonates, polyphosphazenes); and the like.

Furthermore, nicotinamide and its derivatives, analogues, and related compounds can be included in polymer-drug conjugates, including, but not limited to: Vitamin B3 conjugates; hot melt extrudates; Nektar polymer conjugates; pegylates, drug-PEG conjugates; hyaluronic acid derivatives or complexes; HPMA-drug copolymers; and the like. See, Kadajji, et al. (2011), and Rinaudo (2006), and references cited therein.

Particle size of water-soluble and/or water-dispersible polymers also plays an important role. One of the key aspects of Niapads™ is its water-soluble grittiness which helps scrub away oily residues and dead skin cells on acne prone skin without excessive abrasion. A unique combination of grittiness and surfactants (as a blend) help cut through and penetrate oily residues on acne prone skin. This gently abrasive property also makes the substantially dry pads highly effective in dealing with acne. In general, micronized particle sizes of PVP and PEG2M produce less grittiness than other materials, while providing the appropriate degree of grittiness. An average particle size of about 150 microns produces a desirable grittiness in the final product. Particle sizes used to make the final product can range from about 50 microns to about 200 microns.

A useful range for the water-soluble and/or water-dispersible gritty polymer components is from about 10% to about 75% w/w based on the total weight of the formulation. One suitable range for the water-soluble and/or water-dispersible gritty polymer components is from about 30% to about 60% w/w based on the total weight of the formulation. Another suitable range for the water-soluble and/or water-dispersible gritty polymer components is from about 55% to about 65% w/w based on the total weight of the formulation. Another suitable range for the water-soluble and/or water-dispersible gritty polymer components is from about 55% to about 75% w/w based on the total weight of the formulation.

It should be apparent that although grittiness or abrasiveness is a desirable characteristic in the initial and/or final formulations as applied to the substantially dry pad, this property must be moderated for effectiveness, comfort, and ease of use by the patient. When the pad is in use, gentle scrubbing is employed by hand.

One useful water-soluble and/or water-dispersible polymer is PEG-2M (available as POLYOX WSR N-10 from Dow Chemical, Midland, Mich.). PEG 2M is a water-soluble polymer of ethylene oxide. Its functionality is: emulsion stabilizer, viscosity increasing agent. This material is commercially available in various grades with different molecular weights. It can be substituted in the formulation with other grades and/or blend of different grades and/or ingredient(s) and/or cellulose based polymers with similar functionality that are available commercially.

Another useful water-soluble and/or water-dispersible polymer is PVP in one or more of its many forms. For example, PVP is available as an amorphous powder (ISP Corp./Ashland Inc., Covington, Ky.), including products known as, but not limited to, PVP K-15, PVP K-30, PVP K-60, PVP K-90, PVP K-120, and the like. PVP is a water-soluble polymer used as a suspending agent. This material is commercially available in various grades with wide ranging molecular weights, linear, cross-linked, modified grades that impart feel, stability, binding, suspension and viscosity enhancing properties. It can be substituted in the formulation with ingredient(s) with similar functionality and/or blend of grades and/or functionalities that are available commercially.

Exemplary Formulations Having Gritty Water-Soluble Polymers

The following representative formulation TABLES illustrate compositions including nicotinamide.

FORMULATION 1

| Ingredients | Weight (g) |
|---|---|
| Lauryl Lactate | 1.80 |
| Lauryl/Myristyl monoethanoleamide | 0.73 |
| Lavender Oil | 0.92 |
| Macroglyceryl hydroxystearate | 7.32 |
| Methyl Paraben | 0.02 |
| Niacinamide (Vitamin B3) | 4.03 |
| PEG 2M | 23.25 |
| PEG 400 | 13.73 |
| Polyvinyl pyrrolidone | 41.92 |
| Sodium Coco-sulfate | 0.92 |
| Sodium EDTA | 0.04 |
| Sodium lauryl sulfoacetate | 5.49 |
| Water | 400.00 |
| Total | 500.15 |

FORMULATION 2

| Ingredients | Weight (g) |
|---|---|
| Lauryl Lactate | 1.76 |
| Lauryl/Myristyl monoethanoleamide | 0.44 |
| Lavender Oil | 0.70 |
| Macroglyceryl hydroxystearate | 5.94 |
| Methyl Paraben | 0.04 |
| Niacinamide (Vitamin B3) | 4.02 |
| PEG 2M | 44.34 |
| PEG 400 | 10.54 |
| Polyvinyl pyrrolidone | 26.51 |
| Sodium Coco-sulfate | 1.32 |
| Sodium EDTA | 0.04 |
| Sodium lauryl sulfoacetate | 4.39 |
| Water | 400.00 |
| Total | 500.0 |

FORMULATION 3

| Ingredients | Weight (g) |
|---|---|
| Lauryl Lactate | 1.88 |
| Lauryl/Myristyl monoethanoleamide | 0.88 |
| Lavender Oil | 0.75 |
| Macroglyceryl hydroxystearate | 8.85 |
| Methyl Paraben | 0.06 |
| Niacinamide (Vitamin B3) | 4.01 |
| PEG 2M | 55.31 |
| PEG 400 | 6.08 |
| Polyvinyl pyrrolidone | 16.59 |
| Sodium Coco-sulfate | 1.33 |
| Sodium EDTA | 0.06 |
| Sodium lauryl sulfoacetate | 4.20 |
| Water | 400.00 |
| Total | 500.00 |

FORMULATION 4

| Ingredients | Weight (g) |
|---|---|
| Lauryl Lactate | 1.78 |
| Lauryl/Myristyl monoethanoleamide | 2.15 |
| Lavender Oil | 1.15 |
| Macroglyceryl hydroxystearate | 10.10 |
| Methyl Paraben | 0.06 |
| Niacinamide (Vitamin B3) | 4.01 |
| PEG 2M | 60.00 |
| PEG 400 | 11.50 |
| Polyvinyl pyrrolidone | 0.00 |
| Sodium Coco-sulfate | 5.00 |
| Sodium EDTA | 0.06 |
| Sodium lauryl sulfoacetate | 4.20 |
| Water | 400.00 |
| Total | 500.00 |

FORMULATION 5

| Ingredients | Weight (g) |
|---|---|
| Lauryl Lactate | 2.10 |
| Lauryl/Myristyl monoethanoleamide | 3.00 |
| Lavender Oil | 1.14 |
| Macroglyceryl hydroxystearate | 9.80 |
| Methyl Paraben | 0.05 |
| Niacinamide (Vitamin B3) | 4.01 |
| Polyvinyl pyrrolidone (A)* | 25.00 |
| PEG 400 | 11.00 |
| Polyvinyl pyrrolidone (B)** | 34.64 |
| Sodium Coco-sulfate | 5.00 |
| Sodium EDTA | 0.06 |
| Sodium lauryl sulfoacetate | 4.20 |
| Water | 400.00 |
| Total | 500.00 |

*Polyvinyl pyrrolidine (A): PVP-CL (cross-linked).
**Polyvinyl pyrrolidine (B): PVP K-90.

FORMULATION 6

| Ingredients | Weight (g) |
|---|---|
| Lauryl Lactate | 1.78 |
| Lauryl/Myristyl monoethanoleamide | 1.15 |
| Lavender Oil | 1.02 |
| Macroglyceryl hydroxystearate | 10.10 |
| Methyl Paraben | 0.06 |
| Niacinamide (Vitamin B3) | 4.01 |
| PEG 2M | 45.78 |
| PEG 400 | 11.50 |
| Polyvinyl pyrrolidone (B + C)*** | 15.35 |
| Sodium Coco-sulfate | 5.00 |
| Sodium EDTA | 0.06 |
| Sodium lauryl sulfoacetate | 4.20 |
| Water | 400.00 |
| Total | 500.00 |

***Polyvinyl pyrrolidine (B + C): PVP K-90 + PVP K-15 (2:1, w/w)

FORMULATION 7

| Ingredients | Weight (g) |
|---|---|
| Lauryl Lactate | 1.50 |
| Lauryl/Myristyl monoethanoleamide | 1.98 |
| Lavender Oil | 1.00 |
| Macroglyceryl hydroxystearate | 9.10 |
| Methyl Paraben | 0.06 |
| Niacinamide (Vitamin B3) | 4.01 |
| PEG 2M | 54.65 |
| PEG 400 | 6.08 |
| Polyvinyl pyrrolidone | 16.59 |
| Sodium Coco-sulfate | 0.62 |
| Sodium EDTA | 0.06 |
| Sodium lauryl sulfoacetate | 4.35 |
| Water | 400.00 |
| Total | 500.00 |

The aforementioned embodiments in Formulations 1-7 are effective to provide nicotinamide to the skin of the user and produce the desired anti-inflammatory effect The components listed above generally illustrate the range, versatility, and ruggedness of the formulations.

For example, in the food industry, it is well documented that a blend of non-nutritive sweeteners is more effective than individual sweeteners, when it comes to replacing sugar in a given formulation.

In a similar manner, a blend of surfactants and/or emollients and/or film formers acting in a synergistic manner have the potential to perform a better role in fulfilling the objectives as described herein. Thus, various other components and useful additives or excipients may include surfactants, solublizers, humectants, lubricants, wetting agents, suspending agents, stabilizers, emulsifiers, foaming agents, preservatives, anti-microbial agents, and the like.

One useful ingredient is PEG 400. This material is used as a solubilizer and emulsifier. It imparts non-greasy silky-feel and its properties include humectant and lubricant. Polyethylene glycol is commercially available from Dow Chemical (Midland, Mich.) in a wide range of molecular weights and grades. It can be substituted in the formulation with other grades and/or a blend of grades that are available commercially.

Macroglycerol hydroxystearate is also known as PEG-40 castor oil. This material is a good emulsifier, solubilizer and helps impart a smooth skin-feel. It is a useful as humectant, solubilizer for essential oils and is a good lubricant. It is available in other grades with wide range of molecular weights from companies like BASF (Ludwigshafen, Germany). It can be substituted in the formulation with other grades and/or a blend of grades that are available commercially.

Sodium Lauryl Sulfoacetate is a skin friendly surfactant that provides foaming, wetting and emulsification (available from Essential Wholesale & Labs, Portland, Oreg.). This material is a suitable alternative to Sodium Lauryl Sulfate. In the formulation it can be substituted with surfactants with similar properties and/or can be used as a blend with other surfactants with compatible properties, available commercially.

Lauryl Lactate is commercially available from Ashland Specialty Ingredients (Covington, Ky.) and is used as a skin conditioning agent and emollient, e.g., Ceraphyl® 31. The role of lactate esters as skin permeation enhancers in transdermal applications is well documented. Lauryl lactate is chosen for its potential to be able to deliver nicotinamide more effectively. It can be substituted in the formulation with ingredients with similar properties and/or can be used as a blend with other emollients with compatible properties, available commercially.

As discussed herein, the formulations and the substantially dry pads including the formulations are used and applied topically. Typical transdermal delivery requires application of specialized matrices or gels containing an active material, and/or specialized adhesives, and also extended release times (i.e., hours or days of treatment) all in contact with the skin surface. In contrast, the present formulations may be applied generally rapidly, by hand, to the affected skin surface in need of treatment, in many cases in less than one minute, and at longest over several minutes.

Sodium Coco-Sulfate (available from Essential Wholesale & Labs, Portland, Oreg.) is used for foaming and viscosity building and works well in conjunction with other surfactants in the formulation. It can be substituted in the formulation with surfactants with similar properties and/or can be used as a blend with other surfactants with compatible properties, available commercially.

Lauryl/Myristyl Monoethanoleamide is a surfactant with foam boosting and stabilizing properties. This material is commercially available from Sigma-Aldrich (Milwaukee, Wis.). It can be substituted in the formulation with surfactants with similar properties and/or can be used as a blend with other surfactants with compatible properties, available commercially.

Sodium EDTA (available from Dow Chemical, Midland, Mich.) is used as a chelating agent to bind trace metals in the formulation and prevent product degradation or oxidation. This material can be substituted in the formulation with other chelating agents with similar properties and/or can be used as a blend with other chelating agents with compatible properties, available commercially.

Methyl Paraben is used as a preservative and anti-microbial agent. This material can be substituted in the formulation with other anti-microbials with similar properties and/or can be used as a blend with other anti-microbials with compatible properties, available commercially.

Essential oils may be used in the formulation. One particularly useful oil is lavender oil. Lavender essential oil may be obtained from the flowers of Lavendula angustifolia (available from Essential Wholesale & Labs, Portland, Oreg.). This material acts as an emollient and has skin healing properties. In addition, lavender oil has well documented anti-microbial, anti-fungal, anti-eczema, anti-psoriasis and skin soothing properties. These properties make it an ideal ingredient in acne relief and/or acne-treatment product. It can be substituted with other natural oils, botanicals, or extracts with similar properties.

Lavender comes from the Latin root lavare, which means “to wash.” In ancient times, Lavender was frequently used in baths to help purify the body. Since then, Lavender has also been used effectively for a range of ailments such as insomnia, anxiety, depression and fatigue. Herbalists use Lavender oil to treat skin ailments, such as fungal infections (like candidiasis), wounds, eczema, as well as Acne. Lavender is analgesic, anti-microbial, anti-fungal anti-inflammatory and has for centuries been used for its beneficial effect on skin, soothing burns and healing wounds, stimulating the growth of skin cells, treating eczema and psoriasis.

Skin beneficial natural oils such as lavender oil may be used in the substantially dry pads in conjunction with nicotinamide and niacinamide. Other formulations could include witch hazel, rose oil/extract, chamomile oil/extract, Palmarosa oil, Ylang Ylang oil, Tea tree oil, citrus oils such as Bergamot, or grapefruit oil. Aqueous plant extracts including, but not limited to, extracts of cucumber, aloe vera, and the like, can be added.

Exemplary nonwoven pads containing and/or coated with a formulation comprising nicotinamide.

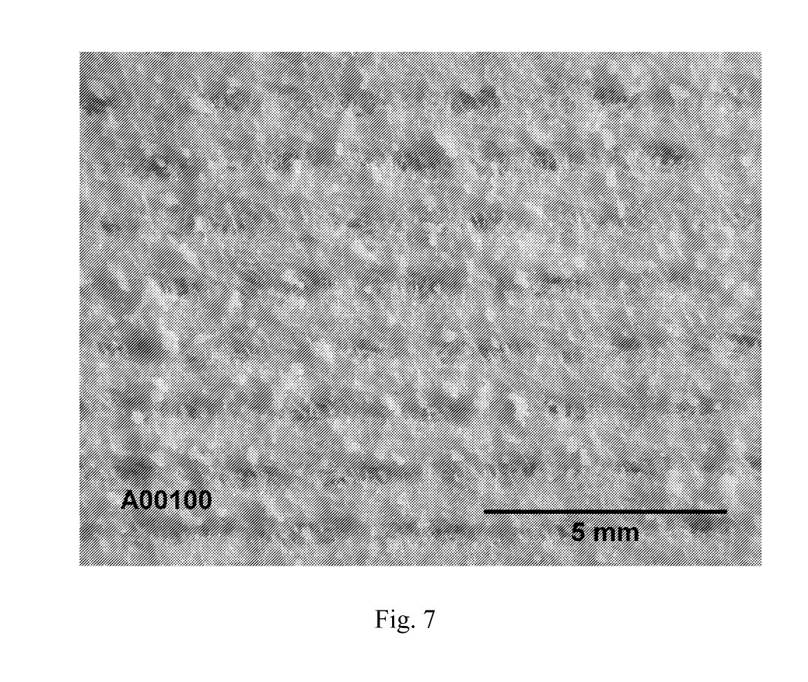

During the development of substantially dry woven or nonwoven pads made in accordance with the principles of the invention, formulations were prepared as described above. FIG. 7 illustrates a typical application of the formulation/coating process to a nonwoven pad (A100). It was noted in preparation of this batch that a relatively thinner coating was applied in comparison with later batch preparations (discussed below). FIG. 7 shows gritty polymer granules adhered to a top surface of the nonwoven substantially dry pad. Partial impregnation of the fibrous matrix by the coating is observed as well. The degree of absorption, incorporation or integration of the formulation into the fibers of the nonwoven material will depend on the coating process parameters during production. Also, the granules have been shown to be the effective agents that help in exfoliation and scrubbing of oily residues from human skin.

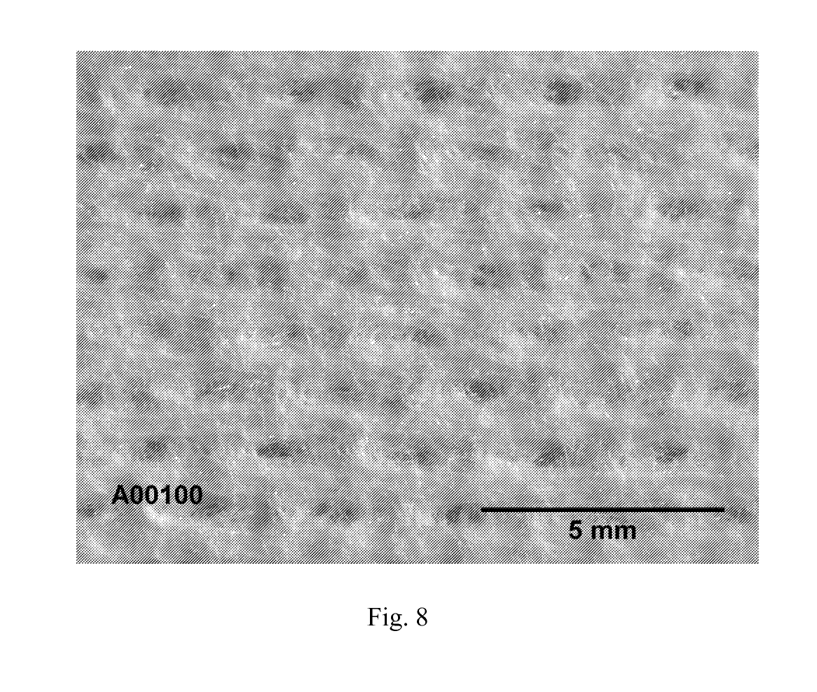

FIG. 8 shows the reverse, i.e., a bottom surface of the nonwoven pad, which does not contain nearly as much of the gritty polymer particles. One difficulty encountered at this stage of development was that a significant portion of the gritty granules would drop off, fall off, and/or scrape off during storage, mostly due to friction and/or lack of attachment or adherence. Such premature removal of gritty polymer granules from the pad is not advantageous since less of the gritty formulation is subsequently available to the user when the pad is applied to the user's skin. Therefore, retention of the gritty polymer particles after incorporation on and in the pads is a desirable feature recognized in the development of the present invention.

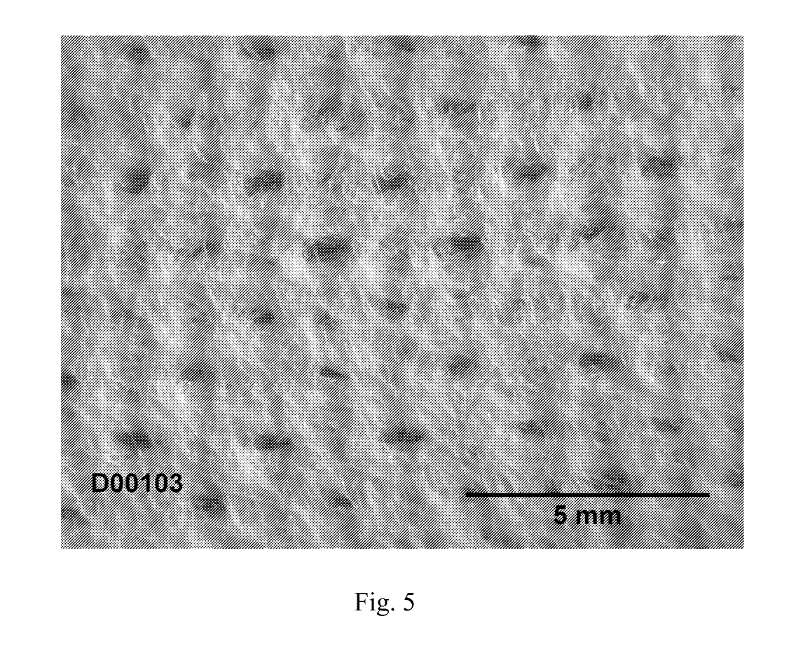

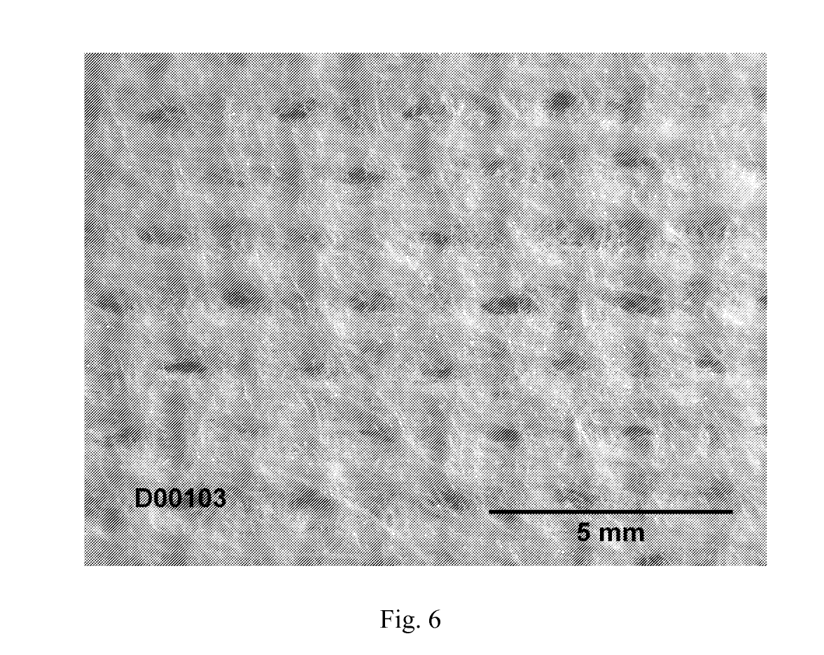

FIGS. 5 and 6 illustrate a formulation that provides a pad lacking visible gritty granules at the magnifications used and providing a generally smooth surface (D103), front and back respectively. User feedback after treatment of human skin indicated that the granular texture was preferred over a smoother texture.

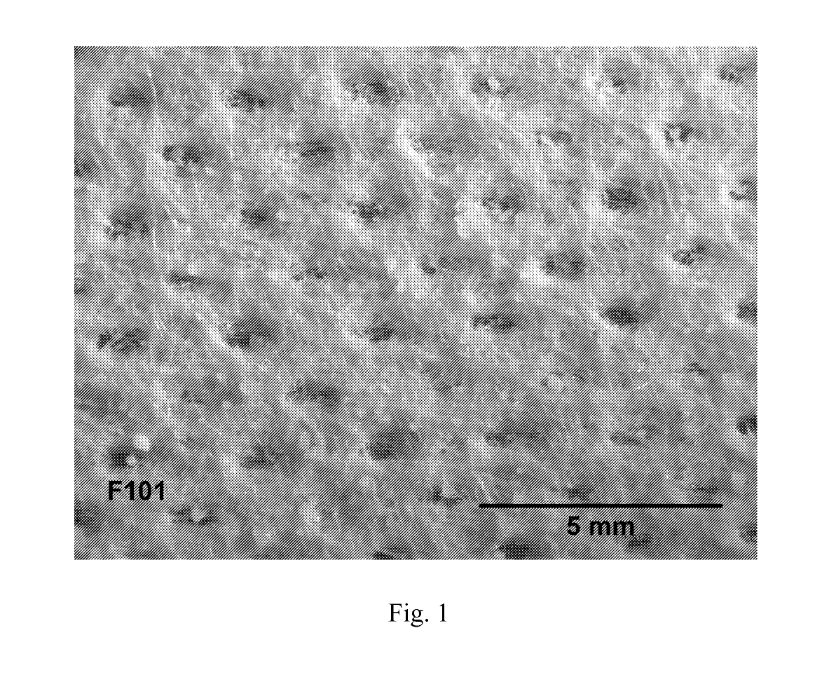

FIG. 1 illustrates a working example prepared in accordance with the present invention (F101). Formulations 1-7 may be applied to a nonwoven matrix to produce a substantially dry nonwoven pad containing a formulation including between about 1%-10% w/w nicotinamide based on the total weight of the formulation.

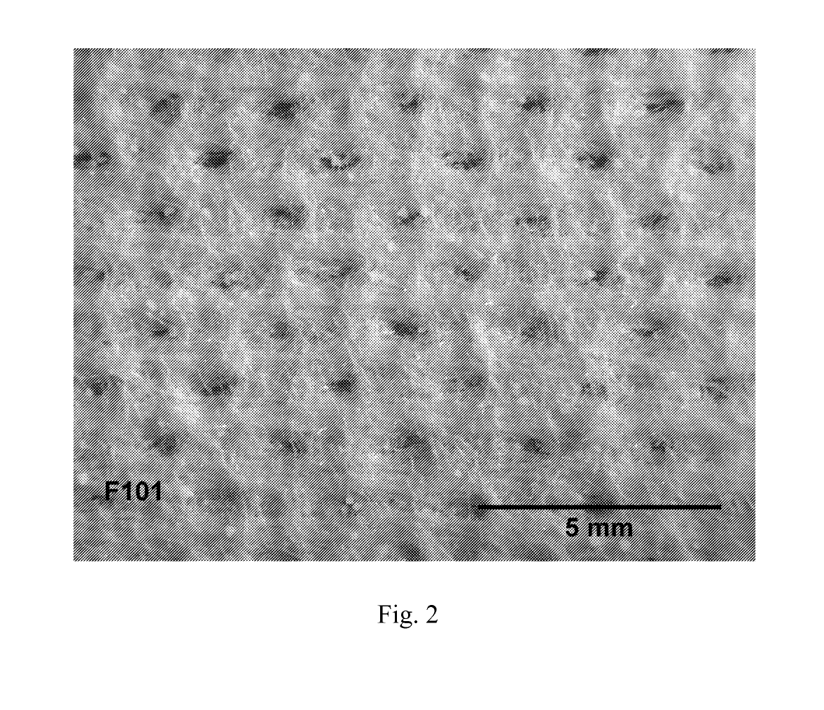

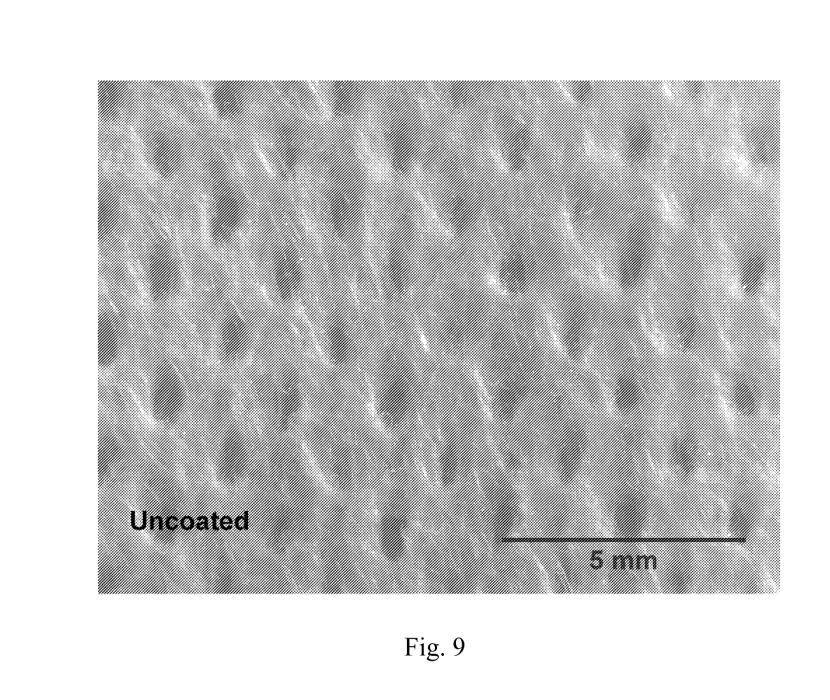

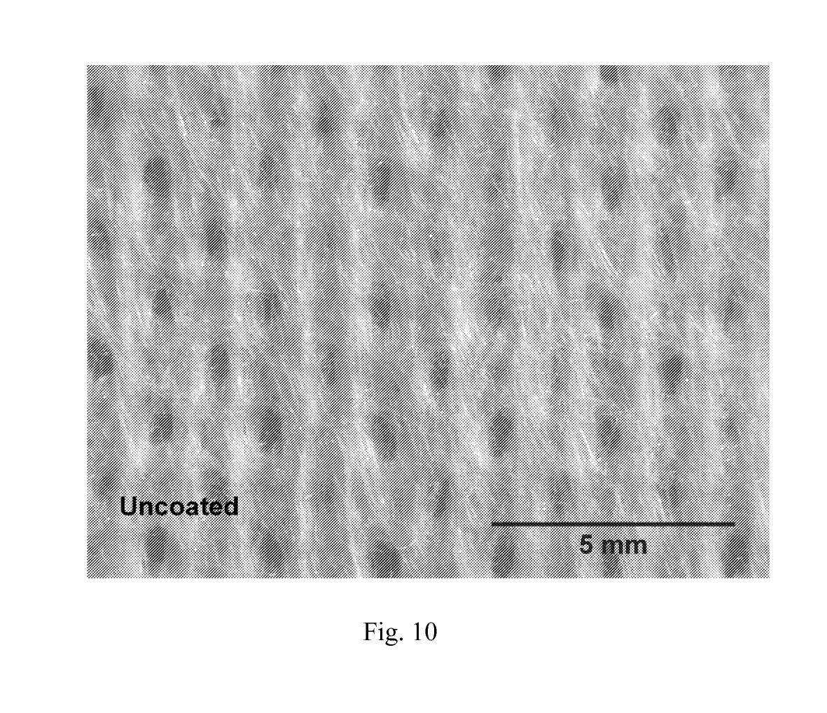

As seen in FIG. 1, the substantially dry nonwoven pad further contains water-soluble gritty polymer in an amount between about 10% to about 75% w/w based on the total weight of the formulation. In an embodiment the at least one water-soluble gritty polymer has an average particle size of from about 50 microns to about 200 microns. The thickness of the coating is thicker than previous batches based on adjustments selected in the production process. FIG. 2 further shows that the granules are substantially adhered to and within the bottom surface of the pad. Overall, increased amounts of gritty polymer granules are present in and on both the front and back surfaces of the pad, and the overall coating is thicker and more desirable when compared to the previous experimental batches. FIGS. 9 and 10 show uncoated, untreated nonwoven pads for comparison.

As described herein, it has been shown that the granules are effective physical and chemical agents that provide exfoliation and scrubbing of oily residues from human skin. Furthermore, the problem of incorporation, deposition, and adherence of the gritty polymer granules to the pad matrix has been discovered and solved herein.

In contrast with previous pad formulations, the described later formulations do not suffer from either granule loss or an applied layer that is too smooth for effective use. In fact, later formulations as disclosed herein and process improvements as described herein demonstrate the effective preparation and use of a substantially dry nonwoven pad containing nicotinamide. FIGS. 3 and 4 depict wider front surface views of the substantially dry nonwoven pad containing nicotinamide and water-soluble gritty polymer made in accordance with the principles of the invention. The preferred formulations and preferred process improvements help to incorporate and retain more exfoliating gritty polymer granules on and in the pads. It is expected that the application of preferred formulations to a woven or nonwoven matrix, material or pad can be fine-tuned to adjust loading levels for the formulation on the pad as desired. It is further expected that the application of preferred formulations to a woven or nonwoven matrix, material or pad can be fine-tuned to adjust coating or layer thickness on the pad as desired.

Most skin care products have essential oil or mild natural, nature identical or artificial fragrance and/or fragrance ingredients. They impart a soothing, calming, or other sensual experience that lingers on the skin each time the product is used. However, some users prefer unscented products for personal or health reasons (including allergy, etc.). For these reasons, an unscented version without Lavender oil was prepared. Since some users may have sensitivities to perfumes and scents, several unscented formulations were prepared and used as follows.

Exemplary Unscented Formulations Having Gritty Water-Soluble Polymers

The following representative formulation TABLES illustrate compositions including nicotinamide.

FORMULATION A

| Ingredients | Weight (g) |
|---|---|
| Lauryl Lactate | 2.30 |
| Lauryl/Myristyl monoethanoleamide | 3.00 |
| Macroglyceryl hydroxystearate | 9.50 |
| Methyl Paraben | 0.05 |
| Niacinamide (Vitamin B3) | 4.01 |
| Polyvinyl pyrrolidone (A)* | 25.00 |
| PEG 400 | 12.30 |
| Polyvinyl pyrrolidone (B)** | 34.58 |
| Sodium Coco-sulfate | 5.00 |
| Sodium EDTA | 0.06 |
| Sodium lauryl sulfoacetate | 4.20 |
| Water | 400.00 |
| Total | 500.00 |

*Polyvinyl pyrrolidine (A): PVP-CL (cross-linked).
**Polyvinyl pyrrolidine (B): PVP K-90.

FORMULATION B

| Ingredients | Weight (g) |
|---|---|
| Lauryl Lactate | 1.78 |
| Lauryl/Myristyl monoethanoleamide | 1.15 |
| Macroglyceryl hydroxystearate | 10.10 |
| Methyl Paraben | 0.06 |
| Niacinamide (Vitamin B3) | 4.01 |
| PEG 2M | 45.20 |
| PEG 400 | 12.52 |
| Polyvinyl pyrrolidone blend (B + C)*** | 15.93 |
| Sodium Coco-sulfate | 5.00 |
| Sodium EDTA | 0.06 |
| Sodium lauryl sulfoacetate | 4.20 |
| Water | 400.00 |
| Total | 500.00 |

***Polyvinyl pyrrolidine (B + C): PVP K-90 + PVP K-15 (2:1, w/w)

FORMULATION C

| Ingredients | Weight (g) |
|---|---|
| Lauryl Lactate | 1.80 |
| Lauryl/Myristyl monoethanoleamide | 0.73 |
| Macroglyceryl hydroxystearate | 7.32 |
| Methyl Paraben | 0.03 |
| Niacinamide (Vitamin B3) | 4.03 |
| PEG 2M | 28.79 |
| PEG 400 | 8.94 |
| Polyvinyl pyrrolidone | 41.92 |
| Sodium Coco-sulfate | 0.92 |
| Sodium EDTA | 0.04 |
| Sodium lauryl sulfoacetate | 5.49 |
| Water | 400.00 |
| Total | 500.00 |

FORMULATION D

| Ingredients | Weight (g) |
|---|---|
| Lauryl Lactate | 1.76 |
| Lauryl/Myristyl monoethanoleamide | 0.44 |
| Polyvinyl pyrrolidone (D)† | 11.52 |
| Macroglyceryl hydroxystearate | 5.94 |
| Methyl Paraben | 0.04 |
| Niacinamide (Vitamin B3) | 4.02 |
| PEG 2M | 44.34 |
| PEG 400 | 10.54 |
| Polyvinyl pyrrolidone (A)* | 15.61 |
| Sodium Coco-sulfate | 1.32 |
| Sodium EDTA | 0.04 |
| Sodium lauryl sulfoacetate | 4.39 |
| Water | 400.00 |
| Total | 500.0 |

†Polyvinyl pyrrolidine (D): PVP K-30.
*Polyvinyl pyrrolidine (A): PVP-CL (cross-linked).

FORMULATION E

| Ingredients | Weight (g) |
|---|---|
| Lauryl Lactate | 1.98 |
| Lauryl/Myristyl monoethanoleamide | 1.53 |
| Macroglyceryl hydroxystearate | 8.85 |
| Methyl Paraben | 0.06 |
| Niacinamide (Vitamin B3) | 4.01 |
| PEG 2M | 55.31 |
| PEG 400 | 6.08 |
| Polyvinyl pyrrolidone | 16.59 |
| Sodium Coco-sulfate | 1.33 |
| Sodium EDTA | 0.06 |
| Sodium lauryl sulfoacetate | 4.20 |
| Water | 400.00 |
| Total | 500.00 |

FORMULATION F

| Ingredients | Weight (g) |
|---|---|
| Lauryl Lactate | 1.50 |
| Lauryl/Myristyl monoethanoleamide | 1.00 |
| Macroglyceryl hydroxystearate | 9.10 |
| Methyl Paraben | 0.06 |
| Niacinamide (Vitamin B3) | 4.01 |
| PEG 2M | 55.18 |
| PEG 400 | 6.08 |
| Polyvinyl pyrrolidone | 16.59 |
| Sodium Coco-sulfate | 0.62 |
| Sodium EDTA | 0.06 |
| Sodium lauryl sulfoacetate | 5.80 |
| Water | 400.00 |
| Total | 500.00 |

The aforementioned unscented embodiments in Formulations A-F are effective to provide nicotinamide to the skin of the user and produce the desired anti-inflammatory effect, while also addressing consumer need or preference.

Method of Preparation for Making a Substantially Dry Pad.

First, a suitable nicotinamide-containing formulation is prepared as follows. In a suitable container equipped with a blender, water is added and the blender is started. Other ingredients (as listed in the above formulations) are added starting from the smallest in quantity (by weight) to the largest. Blender speed is adjusted to maintain adequate mixing. The formulation may optionally be warmed to 40° C., to facilitate faster blending. After the solution or suspension mixture is uniform in consistency, it is ready for coating purposes. The formulation can include a substantial percentage by weight of one or more water-soluble gritty polymers (see Formulations 1-7, A-F).

Next, a woven or nonwoven substrate material is coated using the formulation. Several known methods are available for film coating of the substrate material, including, but not limited to, knife-over-roll coating, gap coating, gravure coating, roll coating, reverse roll coating, slot die coating, extrusion coating, immersion (dip) coating, curtain coating, and air knife coating.

One particularly suitable method for applying the formulation onto a nonwoven fabric is by knife-over-roll coating. This process relies on a coating being applied to the nonwoven substrate on a moving roller which then passes through a gap under a knife (blade, or gap knife). As the coating and roller pass through the gap, the excess coating is scraped off. Thus, coating load, level and/or thickness are controlled and/or varied. The process can be used for high viscosity coatings, such as gritty water-soluble polymers, and can produce high coat weights or loading onto the nonwoven substrate.

Once the nicotinamide-containing, and gritty water-soluble polymer formulation has been applied to the nonwoven substrate, the coated nonwoven is dried to remove water using standard techniques. It is preferred that the coated nonwoven product be dried substantially, namely to at least <5% water by weight, and more preferably less than about 1% by weight of water.

When water or moisture is used or present in the manufacturing process, the resulting coated nonwoven substrate is then dried so that it is substantially free of water. The coated nonwoven substrate can be dried by any means known to these skilled in the art. Nonlimiting examples of known drying means include the use of convection ovens, air drying, radiant heat sources, microwave ovens, forced air ovens, and heated rollers or cans. Also, a combination of various drying methods can be used.

The substantially dried coated nonwoven product is then cut into approx. 5 cm×8 cm pieces and packaged for sale. If needed, pads can be made into any other suitable shape and size based on customer/marketing needs.

In one preferred embodiment, the thickness of the uncoated pad is about 0.24 mm, and the thickness of the dried coated nonwoven pad is about 0.34 mm. In another embodiment, the thickness of the uncoated pad is about 0.45 mm, and it is expected that the thickness of the dried coated nonwoven pad is about 0.55 mm. It is understood that variability may occur in the thickness of commercially available nonwoven materials, which in turn lends to the variability in the thickness of the product coated pads. Generally, the present process may add about 0.1 mm to the overall thickness of the coated pads. A thicker overall coating in excess of 0.1 mm may be applied in the present process, depending upon the initial thickness of the uncoated materials. That is, thicker uncoated pads may lead to even thicker coatings.

The substantially dried coated nonwoven product can be stored in a sealed package. One useful package includes a Ziploc type seal strip in between the seal and the product for added protection. Another useful package includes layers of protection including foil for increased shelf life. Another useful package is foil/polyethylene based high barrier packaging with Ziploc type seal. WVTR (water vapor transmission rate) of <0.005 gt./100 in² over 24 h and OTR (oxygen transmission rate of <0.001/cc/m² over 24 h. In an embodiment, the product in its package may be sterilized using a suitable method such as autoclaving or any other suitable commercially available method.

In an embodiment, the viscosity of the formulations used for preparing the coated nonwovens may be varied or adjusted, based on the types of equipment used, or the coating method employed. Viscosity may be adjusted using mechanical means, stirring, heating, use of chemical additives, or any other means compatible with the formulations and coating methods.

Water soluble gritty texture is provided to the product coating on the nonwoven pads to facilitate better scrubbing of oily residues and opening of clogged skin pores. Once the formulation dries up and dry pads are formed, the texture of the resulting pad is gritty. This grittiness slowly dissolves in presence of water (during skin application) and leaves no insoluble residues. Thus, the overall texture of the resulting pad surface is also gritty, including when it is substantially dried.

The above formulations utilize water as solvent. Aqueous and organic alcohol (C1 alcohols such as methanol, C2 alcohols such as ethanol, C3 alcohols such as isopropyl or propyl alcohol), or polyols based formulations are also contemplated. Other solvents that are water miscible may be employed, provided that processing and removal is compatible with the process of making the nicotinamide-containing substantially dry pads. It is also contemplated that alternative formulations may be prepared for use in other coating methods or alternative methods of manufacture, in order to make the nicotinamide-containing pads.

In an embodiment, the medicated pads can include a pharmaceutical formulation containing nicotinamide in a range of about 5%-25% w/w (as a percentage of the total formulation).

In another embodiment, an alternative medicated pad can be prepared according to the principles of the invention, wherein nicotinamide is substituted by another active ingredient or compound. For example, a substantially dry pad can include an analgesic or anesthetic material such as lidocaine for topical application as described.

In another embodiment, the nicotinamide-containing pads can be attached to a mechanized brush for increased scrubbing efficiency. Product pad size or shape may need to be slightly modified for a brush attachment.

The topical formulations as described may be applied to human skin when contained in a nonwoven pad.

The methods described above may be further understood in connection with the following examples.

In one example, if an individual has acne, they take one pad, moisten it with water and apply it to the affected area. Gentle scrubbing with the pad loosens oil residues and dirt, and also opens clogged skin pores. At the same time, nicotinamide present on the pad is delivered to the area and gets rid of acne bacteria. The user then waits for a brief period, i.e. for at least about 15-20 seconds, and rinses off with water. Alternatively, the user can wait for about 20-30 seconds before rinsing. In a further embodiment, the user can wait for about 60 seconds before rinsing.

In another example, the nicotinamide-containing pad is prepared in accordance with the invention and packaged as above. A user tears open the pouch and opens the ziploc seal. The user removes one pad and moistens it with water. Then the user wets the skin application area with water. The user scrubs the skin application area gently with the moistened pad. The wet pad is discarded after use in the regular trash. The user then waits 20 to 30 seconds and rinses the skin area thoroughly with water. Finally, the user gently pats the skin area dry with a clean towel.

In the above examples, it is expected that treatment of the acne afflicted skin area will result in a visible decrease of skin inflammation within at least about 24 to about 48 hours. Usually, the treatment is effective to visibly decrease skin inflammation in less than 24 hours.

The nicotinamide-containing pad treatment described above may be used twice daily, i.e., early morning and night time before going to bed. The pad contains skin-friendly emollients. Additional non-greasy moisturizers may be used, but only if needed by the user.

It is notable that the nicotinamide-containing pads described herein are safely disposable. In an embodiment, the nicotinamide-containing pads described herein are recyclable. In a further embodiment, the nicotinamide-containing pads described herein are biodegradable.

The pharmaceutical compositions of the present invention may be topically administered in combination with a pharmaceutically acceptable carrier. The active ingredients in such formulations may comprise from 1% by weight to 99% by weight, or alternatively, 0.1% by weight to 99.9% by weight. “Pharmaceutically acceptable carrier” means any carrier, diluent or excipient that is compatible with the other ingredients of the formulation and not deleterious to the user. In accordance with one embodiment, suitable nutraceutically acceptable carriers can include methanol, ethanol, aqueous ethanol mixtures, water, propyl alcohol, isopropyl alcohol, propylene glycol, and combinations thereof.

The cosmetic or cosmeceutical compositions of the present invention may be administered in combination with a nutraceutically acceptable carrier. The active ingredients in such formulations may comprise from 1% by weight to 99% by weight, or alternatively, 0.1% by weight to 99.9% by weight. “Nutraceutically acceptable carrier” means any carrier, diluent or excipient that is compatible with the other ingredients of the formulation and not deleterious to the user. Useful excipients include microcrystalline cellulose, magnesium stearate, calcium stearate, any acceptable sugar (e.g., mannitol, xylitol), and for cosmetic use an oil-base is preferred.

In accordance with certain embodiments, the cosmetic and/or topical pharmaceutical compositions disclosed herein can be provided in the form of an ointment, cream, lotion, gel or other transdermal delivery systems as described in L.V. Allen, Jr., et al., Ansel's Pharmaceutical Dosage Forms and Drug Delivery Systems, 9^{th} Ed., pp. 272-293 (Philadelphia, Pa.: Lippincott Williams & Wilkins, 2011) which is incorporated herein by reference.

The use of the terms “a,” “an,” “the,” and similar referents in the context of describing the presently claimed invention (especially in the context of the claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. Methods may be varied or modified as needed to make the products as described herein. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All references cited herein are incorporated by reference in their entirety. The present invention may be embodied in other specific forms without departing from the spirit or essential attributes thereof and, accordingly, reference should be made to the appended claims, rather than to the foregoing specification, as indicating the scope of the invention.

I claim:

1. A woven or nonwoven pad containing a formulation comprising nicotinamide in an amount between about 1% to about 10% w/w based on the total weight of the formulation and at least one water-soluble gritty polymer in an amount between about 10% to about 75% w/w based on the total weight of the formulation, wherein the pad is substantially dry.

2. The woven or nonwoven pad according to claim 1, wherein the at least one water-soluble gritty polymer has an average particle size of from about 50 microns to about 200 microns.

3. The woven or nonwoven pad according to claim 1, wherein the at least one water-soluble gritty polymer has an average particle size of about 150 microns.

4. The woven or nonwoven pad according to claim 1, wherein the at least one water-soluble gritty polymer is selected from the group consisting of polyvinyl pyrrolidine (PVP), polyethylene glycol (PEG), and combinations thereof.

5. The woven or nonwoven pad according to claim 1, wherein nicotinamide is present in an amount between about 4% to about 5% w/w based on the total weight of the formulation.

6. The woven or nonwoven pad according to claim 1, further comprising an emollient oil.

7. The woven or nonwoven pad according to claim 6, wherein the emollient oil is lavender oil present in an amount from about 0.7% to about 5% w/w based on the total weight of the formulation.

8. The woven or nonwoven pad according to claim 1, wherein the moisture content in the pad is less than 1% by weight.

9. A topical method of treating skin inflammation, comprising the steps of:
(a) providing a woven or nonwoven pad containing a formulation comprising nicotinamide in an amount between about 1% to about 10% w/w based on the total weight of the formulation and at least one water-soluble gritty polymer in an amount between about 10% to about 75% w/w based on the total weight of the formulation, wherein the pad is substantially dry;
(b) moistening the pad with water;
(c) applying the moistened pad to an area of skin in need of treatment;
(d) gently scrubbing the area of skin in need of treatment to apply the formulation to the skin;
(e) allowing the formulation to be absorbed into the skin for a period of at least about 20 sec.;
(f) removing the pad from the skin; and
(e) rinsing the skin with water.

10. The topical method according to claim 9, wherein the at least one water-soluble gritty polymer has an average particle size of from about 50 microns to about 200 microns.

11. The topical method according to claim 9, wherein the at least one water-soluble gritty polymer has an average particle size of about 150 microns.

12. The topical method according to claim 9, wherein the at least one water-soluble gritty polymer is selected from the group consisting of polyvinyl pyrrolidine (PVP), polyethylene glycol (PEG), and combinations thereof.

13. The topical method according to claim 9, wherein nicotinamide is present in an amount between about 4% to about 5% w/w based on the total weight of the formulation.

14. The topical method according to claim 9, further comprising an emollient oil.

15. The topical method according to claim 14, wherein the emollient oil is lavender oil present in an amount from about 0.7% to about 5% w/w based on the total weight of the formulation.

16. The topical method according to claim 9, wherein the moisture content in the pad is less than 1% by weight.

17. The topical method according to claim 9, wherein the skin inflammation comprises a skin condition selected from acne or rosacea.

18. A topical method of treating skin inflammation, comprising the steps of:
(a) providing a first woven or nonwoven pad containing a formulation comprising nicotinamide in an amount between about 1% to about 10% w/w based on the total weight of the formulation and at least one water-soluble gritty polymer in an amount between about 10% to about 75% w/w based on the total weight of the formulation, wherein the pad is substantially dry;
(b) moistening the first pad with water;
(c) applying the first moistened pad to an area of skin in need of treatment;
(d) gently scrubbing the area of skin in need of treatment to apply the formulation to the skin;
(e) removing the first pad from the skin;
(f) providing a second pad as in step (a);
(g) moistening the second pad with water;
(h) pressing the second moistened pad to the area of skin in need of treatment in such a manner that the pad adheres to the skin;
(i) allowing the adhered pad to dry on the skin;
(j) removing the adhered pad from the skin; and
(k) rinsing the skin with water.

19. The topical method according to claim 18, wherein nicotinamide is present in an amount between about 4% to about 5% w/w based on the total weight of the formulation.

20. The topical method according to claim 18, further comprising an emollient oil.

21. The topical method according to claim 20, wherein the emollient oil is lavender oil present in an amount from about 0.7% to about 5% w/w based on the total weight of the formulation.

22. The topical method according to claim 18, wherein the moisture content in the pad is less than 1% by weight.

23. The topical method according to claim 18, wherein the skin inflammation comprises a skin condition selected from acne or rosacea.
